(19) United States
(12) Patent Application Publication
(10) Pub. No.: US 2018/0056082 A1
(43) Pub. Date: Mar. 1, 2018

(54) Magnetic Moxibustion Chair

(71) Applicant: Zhongping Lou, Hangzhou (CN)

(72) Inventor: Zhongping Lou, Hangzhou (CN)

(21) Appl. No.: 15/249,378

(22) Filed: Aug. 27, 2016

(51) Int. Cl. A61N 2/00; A47C 31/00; A61H 39/06

(52) U.S. Cl. CPC A61N 2/002; A61H 39/06; A47C 31/003

(57) ABSTRACT

The invention discloses a device for enhancing magnetic moxibustion therapy that includes a sofa structure with a back support and a cushion seat member. A first array of magnetic are positioned side by side in a media and embedded in the back support, with each of said magnetic bars having its negative pole facing outwardly and a second array of magnetic bars having its negative pole facing upwardly and embedded in the cushion seat member such that the negative magnetic fields penetrate to the user's body. The magnetic bars in the first array are parallel to each other and approximately parallel to the back support's front surface, and the magnetic bars in said second array are parallel to each other and approximately parallel to the cushion seat's top surface.

### FIELD OF THE INVENTION

The present invention generally relates to the field of magnetic therapy. More particularly, the invention relates to a magnetic moxibustion chair which generates magnetic field intensity for enhancing the magnetic therapy effects.

### BACKGROUND OF THE INVENTION

In recent years, magnetic therapy has developed quickly and become a new widely promoted therapeutic method due to extensive adaptive symptoms, significant sanitary effects, non-invasion, without pain, small side effects, safety, reliability, simplicity, and understandability as well as economy, etc. Magnetic therapy is the so-called magnetic field therapy, and is a genetic term to utilize magnetic force or magnetic field for treatment of diseased. Generally, the treatment site is placed in a variable magnetic field, and the magnetism generated by the magnetic pole acts on the human tissues, hereby achieving therapeutic purposes of promoting blood circulation, dispelling blood, reducing swelling, easing pain, diminishing inflammation and analgesia, etc.

In some of the existing magnetic therapy chairs, the magnetic blocks are often disposed on soft fabrics, and are usually arranged with a large spacing, hereby leading to low magnetic field intensity, which is normally smaller than 1500 gauss. This magnetic field has a weak penetrability and can only affect the skin surface. This achieves a poor magnetic therapy effect particularly when it acts on the human or animals. What is needed is an apparatus for enhancing the magnetic therapy effects. This can be done by using magnets positioned in side by side creating a magnetic bar. The magnetic bars embedded in a chair in arrays, along with other assembly materials and components to create a magnetic moxibustion apparatus which generates strong magnetic field intensity and provides magnetic therapy for a user, particularly when the magnets arranged with short spacing.

### SUMMARY OF THE INVENTION

The invention discloses a device for enhancing magnetic moxibustion therapy that includes a sofa structure with a back support member and a cushion seat member, a first array of magnets positioned side by side to create individual magnetic bars are embedded in the back support member, and a second array of magnetic bars embedded in the cushion seat member. The magnetic bars in the first array are parallel to each other and approximately parallel to the back support's front surface, and the magnetic bars in said second array are parallel to each other and approximately parallel to the bottom seat's top surface. A base material is fixed to a substrate, while the substrate is disposed on the back support member and seat cushion sitting member of the magnetic moxibustion sofa apparatus. Therefore, the magnets can be arranged with a shorter spacing while not mutually attracted, hereby generating strong magnetic field intensity and enhancing the magnetic therapy effects.

### First Preferred Embodiment and Best Mode:

A device for enhancing magnetic moxibustion therapy which includes a sofa chair, and an array of multiple bar magnets assembled in a media, such as a silicone rubber tube and fixed to a base material according to a preset spacing and embedded inside the sofa chair is the first preferred embodiment and best mode of the invention. The base material is disposed on to the substrate, while the substrate is disposed on a chair back support and seat cushion support member for sitting.

A first array of magnetic bars embedded in the back support member, with each of the magnetic bars having its negative pole facing outwardly and a second array of magnetic bars having its negative pole facing upwardly are embedded in the bottom seat such that the negative magnetic fields penetrate to the user's body. Further, the multiple bar magnets can be arranged in an array with a shorter spacing while not being mutually attracted, and a strong magnetic field is generated. Thus, this enhances the magnetic therapy effects. In addition, the multiple bar magnets can be arranged on the base material with different spacing configurations while the location of the base material is adjustable on the substrate. This enables different requirements to be met when operating the magnetic moxibustion sofa apparatus particularly with high penetration into the user's back, bottom side.

### BRIEF DESCRIPTION OF THE DRAWINGS

FIG. 1 is an illustration of an isometric view of the magnetic moxibustion apparatus invention. In this illustration, the multiple bar magnets are shown in a first array positioned in the back support of the magnetic moxibustion apparatus invention, which is a sofa structure, and a second array. positioned throughout the cushion sitting member of the sofa chair structure.

FIGS. 2A and 2B are front view illustrations of the back member of the sofa chair structure. In FIG. 2A, the magnetic bars are all the same size and length for equal distribution of magnetic moxibustion therapy on a user's back. In FIG. 2B, the magnetic bars have different lengths, and the strongest magnetic bar is in the middle of the back member of the sofa structure to provide optimal moxibustion therapy on the critical area of the user's back.

FIGS. 3A and 3B are illustrations of the cushion sitting member of the sofa chair structure. In FIG. 3A, the magnetic bars are all the same size and length for equal distribution of magnetic moxibustion therapy on a user's back. In FIG. 3B, the magnetic bars have different lengths, and the shape of the magnet bar array is contoured to a user's behind to provide optimal moxibustion therapy.

FIGS. 4A and 4B are illustrations of the individual magnets positioned inside of the magnetic bars. FIG. 4A represents the magnetic bars in the cushion seating member of the sofa chair structure. FIG. 4B represents the magnetic bars in the back support member of the sofa chair structure. The negative pole side of each individual magnet faces the user's back and behind, while the positive pole side faces away from the user's back and behind.

FIG. 5 is an illustration of each individual magnet which is assembled in a media including a substrate. When several individual magnets are assembled in a media with a substrate, they form the magnetic bars. Each individual magnet has a negative pole and a positive pole. When assembling the magnetic bars, the negative pole is positioned to face the back member of the sofa structure and the cushion seating member of the sofa chair structure to allow magnetic moxibustion therapy for the user.

### DETAILED DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation or restriction of the scope of the invention is thereby intended. Any alterations and further implementations of the principles of the invention as described herein are contemplated as would normally occur to one skilled in the art to which the invention relates.

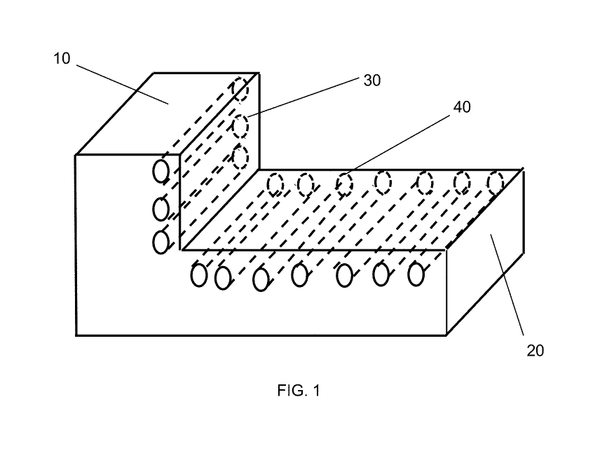

FIG. 1 is the isometric view illustration of the magnetic moxibustion chair apparatus. As it is shown, the magnetic moxibustion chair includes a sofa structure with a back support member 10 and a cushion seat support member 20, but also includes an array of multiple bar magnets 30 on the back support member 10, base material (not shown) and substrate (not shown). Of these, a first array of individual magnets are assembled in a media, such a silicone rubber tube, and embedded in the back support member 10. The individual magnets in the media create the magnetic bars with each of the magnetic bars having its negative pole facing outwardly.

A second array of magnetic bars 40 having its negative pole facing upwardly are also embedded in the cushion seating member 20 of the sofa structure such that the negative magnetic fields can penetrate to the user's body. The magnetic bars in the first array are parallel to each other and approximately parallel to the back support's front surface, and the magnetic bars 40 in said second array are parallel to each other and approximately parallel to the cushion seating member's top surface and fixed to the base material according to the present spacing. The base material is then fixed to the substrate, while the substrate is disposed on the back support member 10 and seat cushion support member 20 of the magnetic moxibustion sofa chair apparatus.

Specifically, the preset spacing may be changed as long as large magnetic field intensity among the multiple magnets can be guaranteed. In this embodiment, the substrate can be made of foam-rubber cushion. In one example, if the substrate is made of foam-rubber cushion, the multiple bar magnets in the back support member 30 can be fixed to the base material, and then the base material is disposed on any position of the foam rubber cushion, which is disposed on the back support member 10 of the sofa chair apparatus and seat cushion support member 20, thereby generating a type of gyromagnetic moxibustion chair with high penetration.

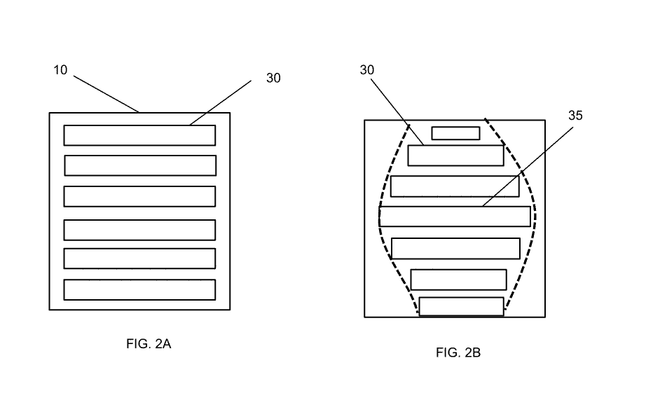

In FIG. 2A, the back support member 10 is shown in a front view. In this view, an array of magnetic bars 30 embedded inside the back support member 10 of the sofa chair apparatus with equal sizes is shown. Each magnetic bar 30 is equal length, width, and height, and each bar is distributed evenly across the surface of the back support member 10. In FIG. 2B, the back support member 10 is shown in a front view, and the array of magnetic bars 30 are of various sizes. The array in FIG. 2B focuses on a user's critical area and the largest magnetic bar 35 is positioned in the middle to provide the optimal moxibustion therapy to the user's critical area, while the upper and low sections of the back support member 10 have smaller magnetic bars.

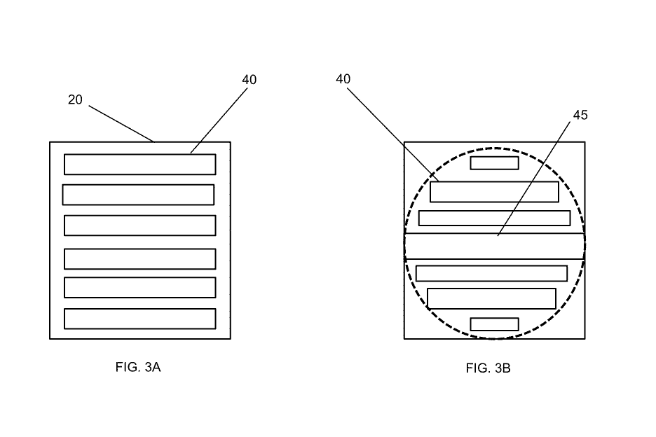

In FIG. 3A, the cushion seating member 20 is shown in a front view. In this view, an array of magnetic bars 40 is embedded inside the cushion seating member 20 of the sofa chair apparatus with equal sizes is shown. Each magnetic bar 40 is equal length. In this embodiment, the base material (not shown) can be sutured or adhered to the substrate. The fixation method is not limited in this embodiment, and can be selected according to the actual application.

Further, since multiple bar magnets are assembled in a media and made of magnetically conductive material, magnetically non-conductive material is selected for the base material (not shown) to avoid interfering with the multiple bar magnets' functionality. A material such as a flexible substrate or hose, etc. can be used for the substrate as well.

FIG. 3A also shows each magnetic bar 40 assembled according to a preset spacing in relation to the other individual bar magnets. Each individual magnet positioned side by side inside a media, such as a silicon rubber tube creates a magnetic bar. The negative poles of each of the individual magnets face upward to enhance magnetic moxibustion therapy for the user when they are seating in the magnetic moxibustion sofa chair apparatus.

Furthermore, referring back to the base material, it can be heated to obtain a thermal-magnetic moxibustion chair with a combination of high-heat and ferromagnetism, which performs hot compression and magnetic therapy to for the user.

During the heating process, the temperature is automatically controlled. In the magnetic moxibustion sofa chair shown in FIG. 1's illustration, the multiple bar magnets 30, 40 are configured in an array and fixed to the base material according to the preset spacing, then the base material is disposed on the substrate, while the substrate is disposed on the chair back support member 10 and seat cushion support member 20. Thus, the multiple bar magnets 30, 40 can be arranged with a shorter spacing while not mutually attracted, hereby generating strong magnetic field intensity. This enhances the magnetic therapy effects. In addition, the multiple bar magnets can be arranged on the base material with different spacing's, and location of the base material is adjustable on the substrate, which enables different requirements to be met, particularly with high penetration methods.

In addition, the preset spacing among the multiple bar magnets 30, 40 is set to 10-30 mm which ensures strong magnetic field intensity. This magnetic field has a strong penetrability and the magnetic lines of force can penetrate into the skin when it acts on the human or animals which improves the microcirculation of the human or animal's bodies. Meanwhile, the multiple bar magnets can be fixed to the base material with a variety of different spacing's, which enables the magnetic moxibustion apparatus to meet different user requirements. Furthermore, after the multiple bar magnets 30, 40 are arranged in an array inside the sofa chair and on the base material, the base material can be disposed on different substrates, and the location of the base material on the substrate is adjustable. Based on this embodiment, the multiple bar magnets can be fixed to the base material according to the unipolar direction but also according to the bipolar direction as well. Of these, arrangements in the unipolar direction means that the N or S poles of the multiple magnets 30, 40 are consistent, while arrangement in the bipolar direction means that the N or S poles of the multiple magnets 30, 40 are inconsistent. Multiple bar magnets 30, 40 are arranged according to the bipolar direction, and they constitute a magnetic force array, in which the adjacent multiple bar magnets 30 , 40 have different polarities, which means S pole is adjacent to N pole, while N pole is adjacent to S pole. The multiple bar magnets 30, 40 are fixed to the base material through multiple grooves, of which one bar magnet is disposed correspondingly in one groove. In addition, the multiple bar magnets 30, 40 can also be fixed to the base material by setting multiple fasteners on the base material, such as buttons. They may also be adhered to the base material. In this example, multiple grooves or multiple buttons are used to fix the multiple bar magnets 30 , 40 to the base material, which can more accurately control the spacing among the array of multiple bar magnets, but also can quickly fix the array of multiple bar magnets to the base material. This also enables the multiple bar magnets to be rapidly and conveniently located.

Furthermore, in another example, the base material is magnetically non-conductive and flexible, which can be a flexible substrate or a hose. If the base material is made of a hose, the array of multiple bar magnets 30, 40 are fixed into the hoses (not shown) according to the preset spacing, and then the hoses are disposed on the substrate, specifically on the back support member 10 or the cushion seat member 20 of the sofa chair structure. In actual application, the multiple bar magnets 30,40 are disposed on the base material according to the requirement of the user, and then the base material can be disposed on different substrates which form magnetic therapy in series. Further, the array of multiple bar magnets 30,40 are disposed into the hoses, and the hoses are disposed on the back support member 10 or cushion seat member 20 of the sofa chair at a distance of 1 inch from the top surfaces to prevent squashing the bar magnets when the user sits down. The magnetic field generated by the multiple bar magnets 30, 40 is larger than 2000 gauss. In this example, when the multiple bar magnets 30 ,40 are fixed to the base material according to the present spacing, the generated magnetic field intensity can be greater than 2000 gauss since the array of multiple bar magnets 30-40 are close to each other in proximity.

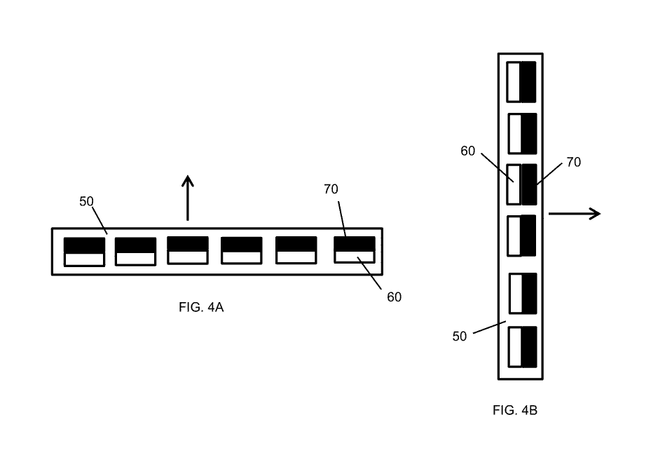

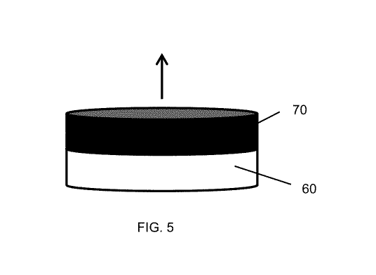

In FIGS. 4A and 4B, one magnetic bar is shown for simplicity. The media includes a substrate material 50 with several individual magnets 60. In FIG. 4A, the negative pole 70 of each individual magnet faces up since FIG. 4A represents one magnetic bar which is positioned in the cushion seat member of the sofa chair apparatus. In FIG. 4B, the negative pole 70 of each of the individual magnets faces toward the right since FIG. 4B represents one magnetic bar which is positioned in the back support member of the sofa chair apparatus. FIG. 5 is the illustration of one individual magnet 60 which is placed in a media. When several individual magnets such as the magnet shown in FIG. 5 are positioned side by side in a media with a substrate, a magnetic bar is created. The negative pole 70 faces out to toward the user's back and bottom area to enhance the magnetic moxibustion therapy when a user is seated in the magnetic moxibustion sofa chair apparatus.

Although one or more embodiments of the newly improved invention have been described in detail, one of ordinary skill in the art will appreciate the modifications to the material selection and magnetic components along with the new footprint layout of the magnetic moxibustion chair. In particular, by using the multiple bar magnets oriented in a preset spacing in a chair, a desirable assembly and more effective footprint is created to enhance magnetic therapy for humans and animals. It is acknowledged that obvious modifications will ensue to a person skilled in the art. The claims which follow will set out the full scope of the claims.

1. An apparatus for enhancing magnetic moxibustion therapy comprising a sofa structure with a back support member and a bottom seating member, a first array of magnetic bars embedded in said back support member and a second array of magnetic bars embedded in said bottom seating member, wherein said magnetic bars in said first array, arranged in one layer, are parallel to each other and to said back support member's front surface, and wherein said magnetic bars in said second array, arranged in one layer, are parallel to each other and to said bottom seating member's top surface, wherein each of said magnetic bars in said first array comprises a number of magnetic pieces, each of which having their negative pole facing outwardly, and wherein each of said magnetic bars in said second array comprises a number of magnetic pieces, each of which having its negative pole facing upwardly.

2. The apparatus of claim 1, wherein said magnetic bars in said first array are identical in length and thickness.

3. The apparatus of claim 1, wherein said magnetic bars in said first array are in different length and thickness.

4. The apparatus of claim 1, wherein each two adjacent magnetic bars in said first array have an identical spacing.

5. The apparatus of claim 1, wherein said magnets in said second array are identical in length and thickness.

6. The apparatus of claim 1, wherein said magnetic bars in said second array are in different length and thickness.

7. The apparatus of claim 1, wherein each two adjacent magnetic bars in said second array have an identical space.

8. The apparatus of claim 1, wherein each two adjacent magnetic bars in said second array have a different spacing.

9. The apparatus of claim 1, wherein all magnetic bars in said first array and said second array are identical.

10. The apparatus of claim 1, wherein each of said magnetic bars in said first array and said second are have different spacing.

11. The apparatus of claim 1, wherein each of said magnetic bars are assembled less than one inch in distance from said back support member and said seating member.

12. The apparatus of claim 1 further comprising a base material and a substrate.

13. The apparatus of claim 13 wherein said base material is fixed to said multiple magnetic bars according to a unipolar and bipolar direction.

14. The apparatus of claim 13 wherein said base material has grooves, and said multiple magnetic bars are affixed to said grooves indented in said base material, and wherein each groove corresponds to one magnetic member.

15. The apparatus of claim 13 wherein said substrate is adhered to said back support member and a bottom seating member.

16. The apparatus of claim 13 wherein said base material is adhered to said substrate.

17. The apparatus of claim 13 wherein said base material is made of silicone rubber tubes, and wherein said multiple magnetic are disposed into said silicone rubber tubes according to a preset spacing.

18. The apparatus of claim 1 wherein said bottom seating member is magnetically non-conductive and flexible.

19. The apparatus of claim 1 wherein the magnetic field intensity generated by said magnetic bars can be greater than 2000 gauss.

20. The apparatus of claim 1 wherein said magnetic pieces are identical.
